(19) United States
(12) Patent Application Publication
(10) Pub. No.: US 2014/0272770 A1
(43) Pub. Date: Sep. 18, 2014

(54) Phototherapy mouthpiece for enhancing the replication of gum cells

(71) Applicant: Edward P. Hurley, Sarasota, FL (US)

(72) Inventor: Edward P. Hurley, Sarasota, FL (US)

(21) Appl. No.: 13/815,706

(22) Filed: Mar. 14, 2013

(51) Int. Cl. A61N 5/06; A61C 5/14

(52) U.S. Cl. CPC A61N 5/0603; A61C 5/14; A61N 5/0613
USPC 433/29

(57) ABSTRACT

A phototherapy mouthpiece includes a mouthguard including a light source and a power pack extending power therethrough a cable to the light source mounted for converting the power to red or near-red photon light, in order to convert the photon energy to proton-motive energy in the gum margin, so that gum cells can replicate to heal gum tissue. The mouthguard portion forms a plurality of recesses and air ducts to enhance oral osmosis. Optionally, a power pack based light source can deliver red or near red photon light through an optic cable to achieve the same proton-motive energy effect, but with less load on the jaw.

### CROSS REFERENCE TO RELATED APPLICATIONS

This is a continuation in part of Ser. No. 13/066,995, filed on Apr. 29, 2011.

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of dentistry and more specifically to a phototherapy mouthpiece for enhancing the replication of gum cells.

There are vast restorative techniques used in dentistry. In some cases, a mouthpiece light source device is worn over the dental arches to light and or heat an oral agent, e.g., tooth whitening agent, tooth filling, etc., in order to administer a restorative response in the mouth. In other times, power pack light source devices are held by a clinician to inspect oral conditions or complete dental work.

Often times, actinic blue light is used to saturate an oral agent for a quick restorative appearance because very little oxygen is present for producing a physical response where there is actinic. Non-actinic light, such as red and near red light, isn't as effective at releasing dental agents to shine of teeth, due to its preferred absorption rate in neighboring soft gum tissue.

One type of light emitting device in the dental industry is disclosed in US Pat. APP. 2008/0233541 A1 ('541) issued Sep. 25, 2008 by Mr. De Vreese. Mr. De Vreese discloses an electroluminescent mouthpiece that is fitted to the teeth and/or gums of a dental arch and used for dental therapy. According to the '541 invention, the mouthpiece is worn with a plurality of illuminating lights often seated longitudinally between the jaw, which is a safety concern because the user can inadvertently bite down on their teeth to possibly crush one or more possible lethal nanolights.

Scientific research shows that the '541 device cannot efficiently replicate gum cells, due to its atomic scale colored 400-660 nanometer spectral frequency inhibiting the interaction of nitric oxide (NO) with hemoglobin (Hb), especially at the 660 nm light spectrum. This nitrogen cycle breakdown slowly dissolves the uptake and delivery of oxygen and saliva to the gums, to thereby promote urea, gum disease and dry mouth. The '541 unit has many obstacles to overcome, including light energy passing through its lens, then lesser light energy passing through its mouthpiece, then at least half the light energy often breaking down a dental agent, then some light energy deflecting back to its lens, then what little energy is left has to maneuver around teeth, etc., to slowly end in the gums. This attenuation of blue photon light discourages the natural healing process of gums to disadvantagely encourage more oral use of synthetic objects. The '541 device suppose to be user friendly, but the monitoring the many different dental agents is a chore for a professional dental authority.

Another typical example of a light emitting device is disclosed in US 2004/0201980 A1 ('980) issued Oct. 14, 2004 by Dan E. Fisher at el. Mr. Fisher discloses a hand-held trans-illumination apparatus for enhancing visibility of oral tissue similar to the way an electron microscope works. In this case, the light spectrum can be adjusted only by a few nanometers to detect hard and soft oral tissue disorders. Healthy and unhealthy gum cells generally absorb the same wavelengths, causing a visible contrast in the unhealthy tissue. In most cases, however, the '980 device must be handheld by the user or clinician for hours, which would be more effective using a sufficient mouthpiece since they require less handling.

In view of the forgoing, there is an ongoing need to provide an improved oral phototherapy device used for delivering mild red or near-red light to the gum margin, in order to convert the photon energy into proton-motive force energy to replicate gum cells and heal gum tissue.

### BRIEF SUMMARY OF THE INVENTION

The primary object of the invention is to provide an oral phototherapy mouthpiece for emitting red or near red photon energy near gum pockets to enhance the replication of gum cells for healing gum tissue.

Another object of the invention is to provide the oral phototherapy mouthpiece providing one or more anterior and posterior walls with a plurality of outer treads intermittent to a plurality of recesses, which aids in vertical osmotic flow of oral fluids at each recess.

Another object of the invention is to provide the oral phototherapy mouthpiece with a plurality of longitudinal air ducts, which aids in longitudinal osmotic flow of oral fluid at each air duct.

Other objects and advantages of the present invention will become apparent from the following descriptions, taken in connection with the accompanying drawings, wherein, by way of illustration and example, an embodiment of the present invention is disclosed.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is an plan view of a mouthpiece light source embodiment of the invention;

FIG. 2 is an exploded view of FIG. 1;

FIG. 3 is a cross sectional view of FIG. 1;

FIG. 4 is a perspective view of a power pack light source embodiment of the invention;

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

### A Problem for Oral Phototherapy Devices:

Some aspects of the present invention provide methods and devices known in the art, such as enhancing oral appearance, general purpose lighting and trans-illumination. The oral phototherapy systems and devices are based on a light source, e.g., lasers, LED's, ELD's, semi-conductors, optical wands, etc., transporting photon energy into the mouth. The use of mouthpiece light source based systems and devices provide a significant advantage over handheld power pack based light source systems and devices because they make it possible to do other activities during the phototherapy session. For example, a mouthpiece light source device can be used, so that the user can multitask, e.g., tie their shoes during oral therapy, etc. On the other hand, the mouthpiece light source systems and devices are bulky bacteria use as a substrate base. Such mouthpiece materials often make it difficult to utilize electronic components outside the mouth. For this reason, some power pack light source systems and devices have the terminal ends of the light source mounted in or near the power pack to deliver a dosage of photon light from near the power pack through an optic cable and to the mouth where a lens distributes the photon light into the mouth, in an attempt to decrease electronic components and or load in the mouth.

### A First Solution for Oral Phototherapy Devices: Mouthpiece Based Light Source Phototherapy

In accordance with the present mouthpiece based light source embodiment of the present invention, there is shown in FIG. 1-3 a phototherapy mouthpiece for treating unhealthy gum tissue generally denoted by number 10 comprising a mouthguard 12 at its use end portion and a power pack 14 at its handling end portion interconnected by an insulated conductor cable 16. Each such mouthguard 12 is sized to fit comfortably over an adult's dental arches; however, the mouthguard 12 can also be shaped as a pacifier (not shown) to fit comfortably and safely in an infant's mouth. Each such mouthguard 12 has a horseshoe shaped base 22 joining a pair of upstanding anterior 23 and posterior 24 walls together which are integrally extended therefrom the base 24. At least one light source 18 including a terminal end portion 19 and a lens end portion 20 mount to the mouthguard 12 anterior wall 23 and or the posterior 24 wall.
To accomplish the important mouthpiece light source function of the invention shown in FIGS. 1-3, each such mouthguard 12 is made of a durable FDA and or ADA recommended material, e.g., medical silicone, polyurethane, etc. The mouthguard 12 anterior wall 23 and or posterior wall 24 can each outwardly include a plurality of treads 26 intermittent to a plurality of recesses 27, in order to enhance vertical osmotic flow of ambient oxygen and saliva at each recess 27 when the mouthpiece light source unit 10 is worn. Thus it is seen that each recess could also be formed into a semi-hourglass shape (not shown) to provide a Venturi effect to increase the vertical oral osmotic flow of fluids, e.g., oxygen, saliva, nitric oxide, CO2, etc., when the user a more troubling gum or dry mouth condition. However, the invention is not so limiting to each tread 26 and each recess 27 being a certain shape or size, but accordingly constructed to enhance oral osmotic flow for the end user. Similarly, the mouthguard 12 is forming, but it is not essential, a plurality of ducts 30 uniformly distributed throughout each tread 26 and the base 22, in order to enhance longitudinal oral osmotic flow of fluids and to lessen some load of the mouthguard 12 on the jaw when the phototherapy mouthpiece 12 is worn. Preferably, each duct 30 is, but it isn't essential also shaped for the Venturi effect.
In accordance with the present invention, FIG. 1-3 shows the mouthguard 12 is hygienically managed at the cable 16; however, it would be appreciated to add a handling unit 32 including a lower housing 33 and an upper housing 34 extending out from the mouthguard 12, so that the handling unit 32 can be simply grasped for the final cleanup technique of the present invention. Preferably, the mouthguard 12 and the handling unit 32 are hinged together so that, when the mouthguard 10 is worn, the handling unit 18 can be pivoted against the person's chin to lessen the mouthguard 12 load on the person's teeth and gums. It should be appreciated, however, that the phototherapy mouthpiece 10 may not be hinged.
Referring again to FIGS. 1-3, the light source 18 is interconnected to an outer 38 strip which is securely mounted to the mouthguard 12 inner anterior wall 23, an inner strip 40 which is securely mounted to the mouthguard 12 posterior wall 24 and or a jumper 41 to fit snugly in a watertight seal. The light source18 has a clear hard covered lens 20 shaped in a sufficient geometric shape, e.g., convex, flat, etc., so as to distribute the mild red or near-red illuminating light uniformly into the gum tissue. It should be appreciated, however, that various other shaped covers and lens can be utilized. Of importance, the light source 18 lens 20 end is situated proximate the gum line while at the same time the light source terminal 19 end is seated significantly away from the mouthguard 12 base 22 and the user's bite, so that the phototherapy mouthpiece 10 cannot be easily damaged and there is little or no interference between the gum-light interaction.
Still referring to FIG. 1-3, the power pack 14 preferably has an electrical power source 42, e.g., four AA button batteries, etc., a controller 44, a ground (not shown) and other electronic parts are mounted in a power pack housing 46, e.g., a two piece housing, etc., to communicate with each light source 18, thereby allowing powering of each light source 18 from the power pack. However, some of the lighter electrical components might be assembled in the mouthpiece 12 and or the handling unit 32 according to sound engineering judgment. The housing 34 has a removable attachable lid 36 for easy handling of the power source 42 at the power station 26 for holding the power source 28, microcircuit 30 and power switch 32 in a watertight seal. Preferably, the housing 34 is removably attached to the power station 18 by a finger sliding track or by other fastener means, e.g., threaded screw 35, etc., for holding each power source 28 in place and for interchange of the power source 28. To be noted, the power pack 14, the mouthpiece 12 and or the handling unit 32 is, but it is not essential, removably attached at the cable 16, so that the controller 44 can be programmed at a dentist's computer to sequence the light source 18 illuminating dosage according to the user's dental exam report. For programming purposes, the controller 44 can be activated one or more times to activate a series of power signals from the power source 42 to the light source 18, to thereby distribute power and light at different gum regions of the mouth. As shown, the controller will generally administer, but it is not essential, the amount of pulse duration, intensity and time of light source 18 illumination dosage at the six upper and lower anterior, mandible and maxilla gum line, to thereby exclude illumination at one of the six non-receding gum regions of the mouth.
As shown in FIG. 1-3, it follows that the mouthpiece light source device 10 based light source 18 is powered by the power pack 14 to deliver mild red light into the gum margin in about the 680 nm, 700 nm, and or 727 nm wavelength spectrum, 4 J/cm̂2 dosage and 20 to 50 mW/cm̂2 radiance for use in healing gum tissue. It also is noted that the light source will deliver, but it is not essential, near-infrared light in the 880 nm wavelength, 4 J/cm̂2 dosage and 20-50 mW/cm̂2 radiance for use in oral pain relief the power pack 14 is turned on at the controller 42 for a predetermined time period to send power to the light source 18, so that the power can be converted to photon energy which can then can be emitted into the person's gum tissues and converted from photon energy to cellular proton motor force energy, which is often called oral bio-stimulus energy or trans-illumination 36, which aids in rotating the gum cells toward the red light to begin the replication of gum cells, to thereby reverse the user's unhealthy gum condition. The controller 44 can have a push button type activator (not shown) and power indicator lights (not shown) centrally positioned on the outer face of the power pack 14 and or the handling unit 18, so as to show the power source 42 is on during the operation and off when the operation has been completed, and the phototherapy mouthpiece 10 can be rinsed and set aside for the next gum treatment.

### The Second Solution for Oral Phototherapy Devices: Power Pack Light Source Based Phototherapy

Turning to FIG. 4, the power pack light source embodiment of the present invention is generally denoted by 10a, which removes electronic components from its mouthguard 12a and mounts them in the power pack 14 and or the handling unit 32. By way of construction, the light source 18 of the type mentioned above has its terminal end portion mounted in the power pack 14 or the handling unit 32 to couple to an optic cable 16a provided with an internal light core 48 at one end, in connection with the optic cable 16a coupling to the light source 18 other lens 20a at the other end. The lens 20a provides a watertight seal to the light source 18 and has, but it is not essential, a wavy outer surface, e.g., corrugated, tread-to-recess, etc., to enhance oral osmotic fluid flow at the concave portion of the wavy surface while acting as an osmotic pump at the convex portion of the wavy surface. The light source 18 other lens 20a includes at least one pair of female catches 50 and the mouthguard12a includes at least one pair of male catchs 52 which removably attach the light source 18a lens 20a to the mouthpiece 12a for use of the power pack optical unit 10a.
Referring to FIG. 4, in practice so that red or near-red light of about the same dosage described above can be emitted at the power pact, then transport through the light core 48 to the lens, with the photon energy converting to proton motive force energy inside the gums. By way of illustration, this power pack phototherapy embodiment 10a can emit light near the gum line in the same dosage as the above mouthpiece embodiment 10a.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. A mouthpiece based light source device for use in oral phototherapy, in order to replicate gum cells to healing gum tissue comprising:
a power pack including a power source;
a controller a conductor cable;
a mouthguard having a base joining an anterior wall and or a posterior wall together for a user to wear over their dental arches;
a light source including a terminal end portion and a lens portion;
wherein the power source interconnects to the controller and one end of the cable at the power pack, in connection with the light source terminal end portion mounting to the anterior wall and or the posterior wall with the light source lens extending out near the gums;
wherein the controller is activated in a predetermined setting to send power from the power pack to the light source to convert the power to mild red light to deliver sufficient mild red light into the gum margin in about the 680 nm, 700 nm, and or 727 nm wavelength spectrum, 4 J/cm̂2 dosage and 20 to 50 mW/cm̂2 radiance, in order to convert the red light to proton-motive force energy in gum cells for repilicating gum cells and healing gum tissue.

2. The mouthpiece based light source device of claim 1, wherein the anterior wall and or the posterior wall of the mouthguard may form a plurality of outer treads intermittent to a plurality of recesses for vertical osmotic flow of oral fluids at each recess.

3. The mouthpiece based light source device of claim 1, wherein the base, the anterior wall and or the posterior wall of the mouthguard may form a plurality of longitudinal air ducts for longitudinal osmotic flow of oral fluid at each air duct.

4. The mouthpiece based light source device of claim 1, wherein a handling unit may extend out from the mouthguard for firmly hygienically handling the unit.

5. The mouthpiece based light source device of claim 4, wherein the handling unit may be hinged to the mouthguard for pivoting the handling unit onto the user's chin during the operation for reducing the load of the mouthguard on the user's jaw.

6. A power pack based light source device for use in oral phototherapy, in order to replicate gum cells to heal gum tissue comprising:
a power pack including a power source and a light source;
a controller an optic cable including a light core;
a mouthguard having a base joining an anterior wall and or a posterior wall together for a user to wear over their dental arches;
a light source including a terminal end portion and a lens end portion;
wherein the power source interconnects to the controller and the terminal end of the light source couples to one end of the cable at the power pack, in connection with the light source the light source lens connecting to the other end of the optic cable and the anterior wall and or the posterior wall facing and beside the gums;
wherein the controller is activated in a predetermined setting to send power from the power pack to the light source to convert the power to mild red light to deliver sufficient mild red light through the light core to the light source lens and into the gum margin in about the 680 nm, 700 nm, and or 727 nm wavelength spectrum, 4 J/cm̂2 dosage and 20 to 50 mW/cm̂2 radiance, in order to convert the red light to proton-motive force energy in gum cells for repilicating gum cells and healing gum tissue.

7. The power pack based light source device of claim 6, wherein a handling unit may extend out from the mouthguard for firmly hygienically handling the unit.

8. The power pack based light source device of claim 7, wherein the handling unit may be hinged to the mouthguard for pivoting the handling unit onto the user's chin during the operation for reducing the load of the mouthguard on the user's jaw.
